(19) United States
(12) Patent Application Publication
(10) Pub. No.: US 2009/0268153 A1
(43) Pub. Date: Oct. 29, 2009

(54) Fixing Structure for Industrial Spectacles and Helmet

(76) Inventor: Tzu-Feng Wang-Lee, Jende Hsiang (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/108,559

(22) Filed: Apr. 24, 2008

(51) Int. Cl. G02C 3/00; A61F 9/00

(52) U.S. Cl. 351/155; 2/10; 2/13

(57) ABSTRACT

A fixing structure for industrial spectacles and a helmet is provided. The industrial spectacles are bilaterally formed with shield portions, each having a hole passing therethrough. Two resilient bands each have a first end formed with a protruding portion for being inserted into the hole of a corresponding one of the shield portions; and a second end for being windingly fastened with a corresponding one of two fasteners, each having at least one through hole and a notch corresponding in shape to a brim extending bilaterally from the helmet. The industrial spectacles bilaterally coupled with the resilient bands are fastened to the helmet by engaging the notch of each of the fasteners windingly fastened to the respective resilient bands with the brim. The industrial spectacles can be pulled down relative to the helmet so as to be used, and pushed up relative to the helmet when not in use.

### BACKGROUND OF THE INVENTION

1. Field of the Invention

The present invention relates to a fixing structure for industrial spectacles and a helmet, and is applicable to workplaces where a worker has to wear a helmet and, when necessary, wear a pair of industrial spectacles as well. The fixing structure for industrial spectacles and a helmet of the present invention enables the industrial spectacles to be resiliently fastened to a brim formed bilaterally on the helmet, pulled down relative to the helmet so as to be used, and pushed up relative to the helmet when not in use.

2. Description of Related Art

Some workplaces require wearing a helmet and even a pair of industrial spectacles when necessary. Workers at the workplaces usually wear commercially available industrial helmets and have to buy and wear additional industrial spectacles that meet the requirement of the workplaces. The industrial spectacles are worn by the workers at work, and are taken off and put aside when no longer required. It is not uncommon for the workers to put aside the industrial spectacles and forget where the industrial spectacles are thereafter. Also, it is inconvenient for a worker to fetch a pair of industrial spectacles placed far from the worker.

### SUMMARY OF THE INVENTION

The present invention overcomes the defects of the prior art regarding existing helmets and industrial spectacles, such as the problem with ease of use. According to the present invention, an assembly design having a new configuration is provided, wherein a pair of industrial spectacles are resiliently fastened to a brim formed bilaterally on a helmet, such that the industrial spectacles can be pulled down relative to the helmet so as to be used, and pushed up relative to the helmet when not in use.

It is a primary objective of the present invention to provide a fixing structure for industrial spectacles and a helmet, wherein the industrial spectacles are bilaterally formed with shield portions, each of which has a hole passing therethrough, while each of two resilient bands, pliable but strong, is formed with a protruding portion at a first end thereof for being tightly inserted into the hole of a corresponding said shield portion of the industrial spectacles. Each of the two resilient bands has a second end for being windingly fastened with a corresponding one of two fasteners, so as to be positioned and form an integral piece therewith. Each said fastener is formed at a first end thereof with at least one through hole, and at a second end thereof with a notch corresponding in shape to a brim extending bilaterally from the helmet. The industrial spectacles bilaterally coupled with the resilient bands are fastened to the helmet by directly engaging the notch of each of the fasteners windingly fastened to the second ends of the respective resilient bands with the brim extending bilaterally from the helmet, thereby allowing convenient assembly of the industrial spectacles with the helmet. In addition, the industrial spectacles can be pulled down relative to the helmet so as to be used, and pushed up relative to the helmet when not in use.

It is another objective of the present invention to provide the aforementioned fixing structure for the industrial spectacles and the helmet, wherein each of the resilient bands coupled to the holes formed bilaterally on the industrial spectacles has a plurality of irregularly shaped marks on both sides of the second end thereof, so as to enable precise adjustment in winding the resilient bands through the through holes of the fasteners and enhance tightness of the winding.

### BRIEF DESCRIPTION OF THE DRAWINGS

FIG. 1 is a perspective exploded view of an industrial spectacles assembly according to the present invention;

FIG. 2 is a perspective view showing the industrial spectacles assembly according to the present invention fastened to a helmet bilaterally; and

FIG. 3 is a schematic drawing showing the industrial spectacles assembled with the helmet when the industrial spectacles are to be used.

### DETAILED DESCRIPTION OF THE PREFERRED EMBODIMENT

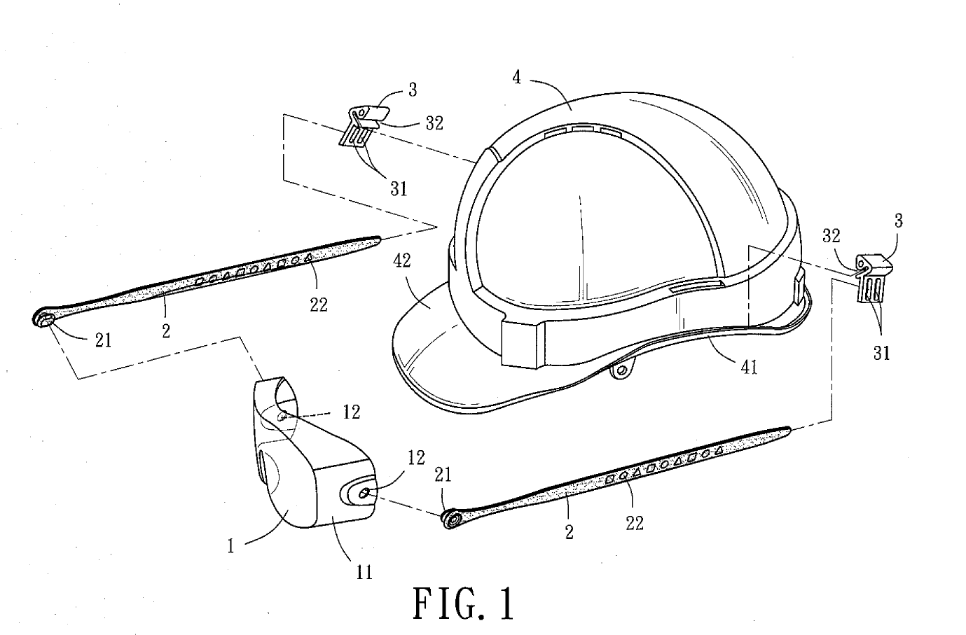

Referring to FIG. 1, the present invention provides a fixing structure for industrial spectacles and a helmet, comprising industrial spectacles 1, two resilient bands 2, two fasteners 3, and a helmet 4.

The industrial spectacles 1 are bilaterally formed with shield portions 11. Each of the shield portions 11 has a hole 12 passing therethrough. (The industrial spectacles can be replaced with tinted welding goggles as appropriate.)

The two resilient bands 2 are pliable but strong enough to provide resilient fastening. A protruding portion 21 constantly larger than each of the holes 12 on the shield portions 11 is formed at a first end of each of the resilient bands 2. Each said resilient band 2 has a second end which is formed on both sides thereof with a plurality of irregularly shaped marks 22. Each said protruding portion 21 is tightly insertable into a corresponding one of the holes 12 formed bilaterally on the industrial spectacles 1.

Each of the two fasteners 3 has a first end formed with at least one through hole 31, so that each of the resilient bands 2 can be wound therethrough. Each of the two fasteners 3 has a second end formed with a notch 32 corresponding in shape to a brim 41 extending from the helmet 4 bilaterally.

The helmet 4 is bilaterally formed with the brim 41 and anteriorly formed with a visor 42 extending forward.

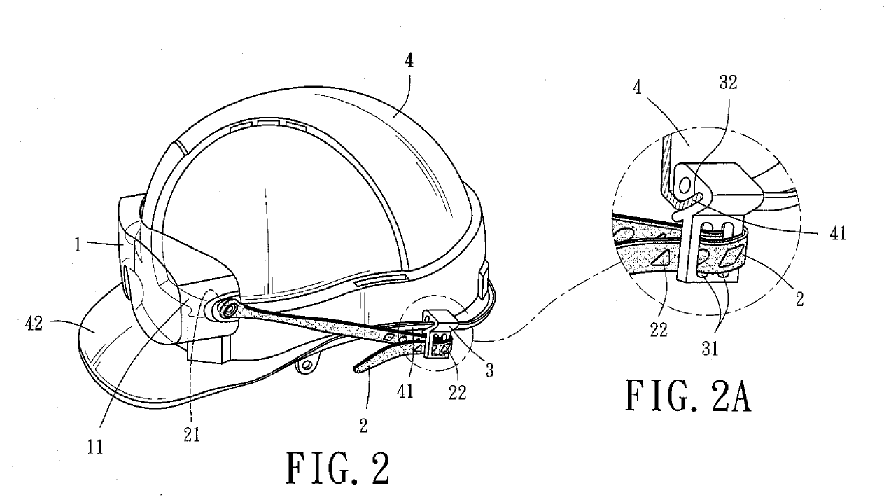

Referring to FIG. 1 and FIG. 2, the industrial spectacles 1, the two resilient bands 2, the two fasteners 3, and the helmet 4 are assembled in the following manner. The protruding portion 21 formed at the first end of each of the resilient bands 2 is inserted tightly into the hole 12 formed on a corresponding one of the shield portions 11 flanking the industrial spectacles 1, wherein the protruding portion 21 is tightly inserted into the hole 12 of each said shield portion 11 via deformation of the resilient bands 2. Then, the second end of each of the resilient bands 2 is wound through the at least one through hole 31 formed at the first end of a corresponding one of the fasteners 3, wherein the irregularly shaped marks 22 formed on both sides of the second end of each said resilient band 2 enable precise adjustment in winding the resilient bands 2 and enhances tightness of the winding, as shown in FIG. 2A. Finally, the industrial spectacles 1 bilaterally coupled with the resilient bands 2 are fastened to the helmet 4 by directly engaging the notch 32 of each of the fasteners 3 wound with the second ends of the resilient bands 2 with the brim 41 extending bilaterally from the helmet 4, as shown in FIG. 2A, so that the industrial spectacles 1 and the helmet 4 stably form an integral piece, as shown in FIG. 2.

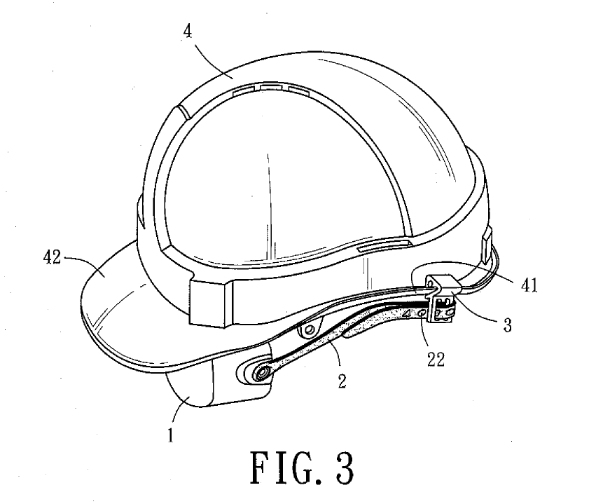

Referring to FIG. 2, the industrial spectacles 1 assembled to the helmet 4 can be pushed up and moved to above the visor 42 of the helmet 4 when not in use. When it is required to use the industrial spectacles 1, referring to FIG. 3, they can be pulled outward and moved to below the visor 42 of the helmet 4 so as to be positioned and thus conveniently worn in front of a worker's eyes. During a break, the worker can move the industrial spectacles 1 up and have them positioned above the visor 42 of the helmet 4 (as shown in FIG. 2) without having to worry that he or she may forget where the industrial spectacles 1 are when it is required to wear the industrial spectacles 1 again. It should be noted that the industrial spectacles 1 or tinted welding goggles are wide enough to enclose a pair of vision correction eyeglasses.

Fastening a pair of industrial spectacles to a brim formed bilaterally on a helmet according to the present invention has the following advantages:

1. The industrial spectacles fastened to the brim formed bilaterally on the helmet are characterized by a simple structure and speedy assembly, wherein the fasteners coupled bilaterally to the industrial spectacles can be engaged with the brim bilaterally formed on the helmet in a convenient manner;

2. The industrial spectacles fastened to the brim formed bilaterally on the helmet can be pushed up and fixed in position above the visor of the helmet while not in use, and pulled down to in front of a user's eyes so as to be used.

3. The industrial spectacles fastened to the brim formed bilaterally on the helmet may be conveniently replaced with ordinary goggles or tinted welding goggles as appropriate.

What is claimed is:

1. A fixing structure for industrial spectacles and a helmet, comprising the helmet and the industrial spectacles, characterized in that:
the industrial spectacles are bilaterally formed with shield portions, each of which shield portions has a hole passing therethrough, while each of two resilient bands, pliable but strong, is formed with a protruding portion at a first end thereof for being tightly inserted into the hole of a corresponding said shield portion of the industrial spectacles, wherein each of the two resilient bands has a second end for being windingly fastened with a corresponding one of two fasteners, so as to be positioned and form an integral piece therewith, in which each said fastener is formed at a first end thereof with at least one through hole, and at a second end thereof with a notch corresponding in shape to a brim extending bilaterally from the helmet;
wherein the industrial spectacles bilaterally coupled with the resilient bands are fastened to the helmet by directly engaging the notch of each of the fasteners windingly fastened to the second ends of the respective resilient bands with the brim extending bilaterally from the helmet, so that the industrial spectacles are conveniently assembled with the helmet into an integral piece, and that the industrial spectacles can be pulled down relative to the helmet so as to be used, and pushed up relative to the helmet when not in use.

2. The fixing structure for the industrial spectacles and the helmet of claim 1, wherein each of the resilient bands coupled to the holes formed bilaterally on the industrial spectacles has a plurality of irregularly shaped marks on both sides of the second end thereof, so as to enable precise adjustment in winding the resilient bands through the fasteners and enhance tightness of the winding.
